(19) United States
(12) Patent Application Publication
(10) Pub. No.: US 2022/0284824 A1
(43) Pub. Date: Sep. 8, 2022

(54) POSTURE AND MOVEMENT IMPROVEMENT SYSTEMS AND METHODS

(71) Applicant: PSYCHOFLIFE LLC, Tucson, AZ (US)

(72) Inventors: Nadav Gerson, Tucson, AZ (US); Jan Halozan, Ptuj (SI); Avinash Baskaran, Plano, TX (US)

(21) Appl. No.: 17/637,288

(22) PCT Filed: Aug. 21, 2020
(86) PCT No.: PCT/US20/47422

§ 371 Date: Feb. 22, 2022

Related U.S. Application Data
(60) Provisional application No. 62/891,157, filed on Aug. 23, 2019.

(51) Int. Cl. G09B 5/02; G09B 5/04; A61B 5/11; A61B 5/00

(52) U.S. Cl. CPC G09B 5/02; G09B 5/04; A61B 5/1124; A61B 5/7425; A61B 5/7455; A61B 2562/0219

(57) ABSTRACT

A method for monitoring and providing feedback regarding motion of a device associated with motion of a user may include displaying a training motion for a user to perform, sensing motion of the user, storing data relating to topography of the user's body based on the user's motion, displaying an activity motion for a user to perform, sensing further motion of the user, storing data relating to the further motion, and comparing the data to provide feedback regarding accuracy of the user's motion. A method for monitoring and providing feedback regarding a user's posture may include periodically sampling a sensor to determine the orientation of the device, determining a proportion of time the device is vertical or nearly vertical, and periodically providing feedback regarding the proportion of time. Systems for performing the methods are also disclosed herein.

### CROSS-REFERENCE TO RELATED APPLICATION

The present application claims priority to U.S. Provisional Patent Application No. 62/891,157, filed Aug. 23, 2019, which is incorporated herein by reference in its entirety.

### TECHNICAL FIELD

The present disclosure is related to posture improvement systems and methods. In particular, the present disclosure is directed to methods and computing systems for analyzing a user's movement of a mobile device related to the user's posture and/or range of motion.

### BACKGROUND

Neck and back pain due to use of technology, such as mobile devices like phones and tablets, is becoming more common. Such pain may be called “tech neck” and may be associated with short- or long-term injuries and reduced productivity. For example, the mass of an average human adult head is approximately ten to twelve pounds. In a neutral position, known as “plumbline posture.” in which a person's ears generally align in a common plane with a user's shoulders as they retract their scapulas, there is relatively little stress on the neck. However, as a person's head protracts (flexes or bends forward), the stress of the head's mass on the spine increases. Because using mobile technology typically involves looking down and away, use of such technology can enable and promote poor posture and may create or exacerbate pain or injury. Some existing wearable devices attempt to address posture improvement, but they are specialized devices that must be worn and must focus on a single body part or preferred posture. Further, existing wearable devices can require a plurality of components (such as a sensor and a separate device to interface with the sensor). In addition, existing devices fail to adequately address use of movement as a pain or injury management tool.

### BRIEF DESCRIPTION OF THE DRAWINGS

Many aspects of the present technology can be better understood with reference to the following drawings. The components in the drawings are not necessarily drawn to scale. Instead, emphasis is placed on illustrating the principles of the present technology. Furthermore, components can be shown schematically in certain views for clarity of illustration only.

FIG. 1 is a block diagram illustrating an overview of devices on which some implementations of the present technology can operate.

FIG. 2 is a block diagram illustrating an overview of an environment in which some implementations of the present technology can operate.

FIG. 3 illustrates a schematic view of a device on which some implementations of the present technology can operate.

FIGS. 4A, 4B, and 4C illustrate side profile views of a user engaging with a device.

FIG. 5 is a flow diagram illustrating a process used in some implementations of the present technology for posture improvement systems and methods.

FIG. 6 is a flow diagram illustrating a process used in some implementations of the present technology for mapping a user's body topography, monitoring a user's motions, comparing the motions to previous motions or to an ideal motion template, and providing feedback.

FIGS. 7A-7E illustrate representative training motions in accordance with implementations of the present technology.

FIGS. 8A-8F illustrate example activity motions that can be used in implementations of the process shown in FIG. 6.

FIGS. 9A-9C illustrate additional example activity motions that can be used in implementations of the process shown in FIG. 6.

FIGS. 10A-10C illustrate additional activity motions in which a user traces a shape with a device, with a focus on wrist movement.

FIGS. 11A-11F illustrate thumb and finger activity motions for implementations of the present technology.

FIG. 12 illustrates a user's deviation in terms of a distance from an ideal or pre-set motion path.

FIG. 13 is a flow diagram illustrating a process used in some implementations of the present technology for monitoring and providing feedback about activity motions, in accordance with aspects of the process shown in FIG. 6.

FIG. 14 illustrates a process for analyzing activity motions with a focus on wrist movement, such as the activity motions described regard to FIGS. 10A-10C.

FIG. 15 illustrates a process for analyzing activity motions on a stable or fixed device with the user navigating their fingers on the display (for example, activity motions described above with regard to FIGS. 11A-11F).

FIG. 16 illustrates a device with a display showing feedback in accordance with some implementations of the present technology.

FIGS. 17A, 17B, and 17C illustrate activity motions that may be accomplished on a stationary device, in a manner similar to the motions described herein with regard to FIGS. 11A-11F.

FIG. 18 is a schematic diagram of various implementations of the present technology.

The techniques introduced here may be better understood by referring to the following Detailed Description in conjunction with the accompanying drawings, in which like reference numerals indicate identical or functionally similar elements.

### DETAILED DESCRIPTION

The present technology is directed generally to posture and movement improvement systems and methods. Systems and methods according to embodiments of the present technology facilitate analysis of a user's posture or motion, provide feedback about the posture or motion, and encourage improvement in the user's posture or motion to alleviate/reduce neck and back pain of the user. The present technology may be implemented in a mobile software application on a portable device, including a mobile communication device such as a smartphone or tablet. Systems configured in accordance with embodiments of the present technology can include a dedicated or discrete portable device having a display, suitable sensors, and a suitable processor or controller programmed with instructions to carry out various process according to embodiments of the present technology.

In one embodiment, for example a method for monitoring and providing feedback regarding motion of a device associated with motion of a user may include displaying a training motion for a user to perform, sensing motion of the user, storing data relating to topography of the user's body based on the user's motion, displaying an activity motion for a user to perform, sensing further motion of the user, storing data relating to the further motion, and comparing the data to provide feedback regarding accuracy of the user's motion. In another embodiment, a method for monitoring and providing feedback regarding a user's posture may include periodically sampling a sensor to determine the orientation of the device, determining a proportion of time the device is vertical or nearly vertical, and periodically providing feedback regarding the proportion of time. Further embodiments include systems for performing methods in accordance with the present technology.

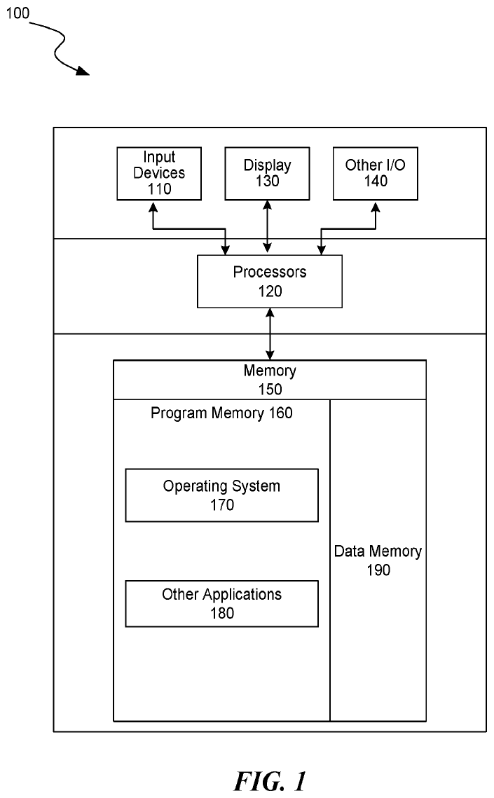

Various embodiments of the technology are described herein. The following description provides specific details for a thorough understanding and an enabling description of these embodiments. One skilled in the art will understand, however, that the technology may be practiced without many of these details. Additionally, some well-known structures or functions, such as those associated with gyroscopes, accelerometers, processors, memory, displays, or mobile devices, may not be shown or described in detail for efficiency and to avoid unnecessarily obscuring the relevant description of the various embodiments. Accordingly, the technology may include other embodiments with additional elements or without several of the elements described below with reference to FIGS. 1-18.

The terminology used in the description presented below is intended to be interpreted in its broadest reasonable manner, even though it is being used in conjunction with a detailed description of certain specific embodiments of the technology. Certain terms may even be emphasized below; however, any terminology intended to be interpreted in any restrictive manner will be overtly and specifically defined as such in this detailed description section.

Specific details of several embodiments of the present technology are described herein with reference to human posture and range of motion. The technology can also be implemented in other applications, including, for example, recreational activities such as sports or yoga.

### A. GENERAL DESCRIPTION OF SUITABLE ENVIRONMENTS IN WHICH THE PRESENT TECHNOLOGY MAY BE IMPLEMENTED

The following discussion provides a general description of a suitable environment in which the present technology may be implemented. Although not required, aspects of the technology described herein can be provided in the form of tangible and non-transitory machine-readable medium or media (such as a hard disk drive, hardware memory, etc.) having instructions recorded thereon for execution by a processor or computer. For ease of description, the combination of a medium or media and a processor or computer can be called a controller, which may be programmed with instructions to carry out a method or process. The set of instructions can include various commands that instruct the computer or processor to perform specific operations such as the methods and processes of the various embodiments described here. The set of instructions can be in the form of a software program or application. The computer storage media can include volatile and non-volatile media, and removable and non-removable media, for storage of information such as computer-readable instructions, data structures, program modules or other data. The computer storage media can include, but are not limited to, random access memory (RAM), read-only memory (ROM), erasable programmable read-only memory (EPROM), electrically erasable programmable read-only memory (EEPROM), flash memory or other solid-state memory technology, compact disc read-only memory (CD-ROM), digital video disc (DVD), or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

Aspects of the technology can also be practiced in distributed computing environments where tasks or modules are performed by remote processing devices, which are linked through a communication network (e.g., a wireless communication network, a wired communication network, a cellular communication network, the Internet, or a short-range radio network (e.g., via Bluetooth). In a distributed computing environment, program modules may be located in both local and remote memory storage devices. Those skilled in the relevant art will recognize that portions of the technology may reside on a server computer, while corresponding portions reside on a client/user computer. Data structures and transmission of data particular to aspects of the technology are also encompassed within the scope of the present technology.

FIG. 1 is a block diagram illustrating an overview of devices on which some implementations of the present technology can operate. The devices can comprise hardware components of a device 100 that facilitates posture and movement analysis and improvement. For example, the devices can include handheld devices, portable or mobile devices such as smartphones or tablets, or other devices with handheld elements. A device 100 can include one or more input devices 110 that provide input to one or more processors 120 (e.g. CPU(s), GPU(s), HPU(s), etc.) notifying the one or more processors 120 of actions and carrying out instructions according to implementations of the present technology. The actions can be mediated by a hardware controller that interprets the signals received from the input device and communicates the information to the one or more processors 110 using a communication protocol. Input devices 110 include, for example, a mouse, a keyboard, a touchscreen, an infrared sensor, a touchpad, a wearable input device, a camera- or image-based input device, a microphone, or other user input devices.

The one or more processors 120 can include single processing units or multiple processing units in a device or distributed across multiple devices. Processors 120 can be coupled to other hardware devices, for example, with the use of a bus, such as a PCI bus or SCSI bus. The processors 120 can communicate with a hardware controller for devices, such as for a display 130. The display 130 can be used to display text and graphics. In some implementations, the display 130 provides graphical and textual visual feedback to a user. In some implementations, the display 130 includes an input device 110 as part of the display, such as when the input device is a touchscreen. In some implementations, the display is separate from the input device. Examples of display devices are: an LCD display screen, an LED display screen, a projected, holographic, or augmented reality display (such as a heads-up display device or a head-mounted device), and so on. Other input/output (I/O) devices 140 can also be coupled to the processor, such as an audio output device (such as one or more speakers), audio input device (such as one or more microphones), one or more cameras, one or more accelerometers (such as one or more three-axis accelerometers), one or more gyroscopes (such as one or more three-axis gyroscopes), global positioning system (GPS) receivers, inertial measurement units, or other sensors for sensing position or movement.

In some implementations, the other I/O devices can include a communication device capable of communicating wirelessly or wire-based with a network node. The communication device can communicate with another device or a server through a network using, for example, TCP/IP protocols or other wired or wireless protocols. The device 100 can utilize the communication device to distribute operations across multiple network devices.

The one or more processors 120 can have access to a memory 150 in a device or distributed across multiple devices. A memory includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs. DVDs, magnetic storage devices, tape drives, and so forth. A memory is not a propagating signal divorced from underlying hardware; a memory is thus non-transitory. The memory 150 can include program memory 160 that stores programs and software, such as an operating system 170 and application programs 180, such as application programs containing and carrying out instructions associated with implementations of the present technology. The memory 150 can also include data memory 190, for example, position or motion data associated with implementations of the present technology, or other data associated with implementations of the present technology, configuration data, settings, user options or preferences, or other data, which can be provided to the program memory 160 or any element of the device 100. Memory can be local and contained within the device 100 or it can include remote storage, such as in or on a server or other computer (such as cloud storage via network computing).

Although implementations of the present technology are described below as being performed in association with a mobile device (such as a smartphone) or a tablet, some implementations can be operational with numerous other computing system environments or configurations. Examples of computing systems, environments, or configurations that may be suitable for use with the technology include, but are not limited to, computers, handheld or laptop devices, wearable electronics, gaming consoles, multiprocessor systems, microprocessor-based systems, programmable consumer electronics, distributed computing environments that include any of the above systems or devices, or the like.

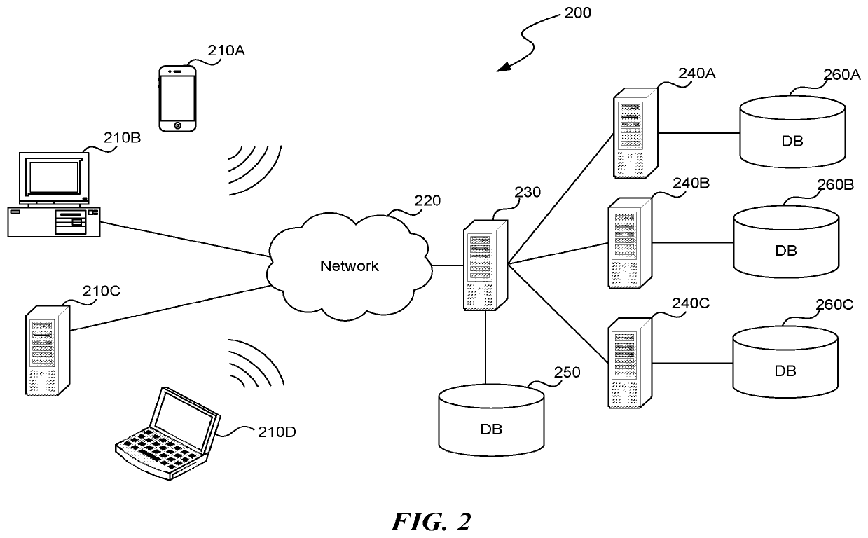

FIG. 2 is a block diagram illustrating an overview of an environment 200 in which some implementations of the disclosed technology can operate. The environment 200 can include one or more client computing devices 210A-D, examples of which can include the device 100 described above with regard to FIG. 1. The client computing devices 210A-D can operate in a networked environment using logical connections through a network 220 to one or more remote computers, such as a server computing device.

In some implementations, a server 230 can be an edge server which receives client requests and coordinates fulfillment of those requests through other servers, such as servers 240A-C. Server computing devices 230 and 240A-C can comprise computing systems, such as the device 100 described above with regard to FIG. 1. Though each server computing device 230 and 240A-C is displayed logically as a single server, server computing devices can each be a distributed computing environment encompassing multiple computing devices located at the same or at geographically disparate physical locations. In some implementations, each server 230, 240A-C corresponds to a group of servers.

Client computing devices 210A-D and server computing devices 230 and 240A-C can each act as a server or client to other server/client devices. The server 230 can connect to a database 250. Servers 240A-C can each connect to a corresponding database 260A-C. Each of the servers can share a database or can have their own database. Databases 250 and 260A-C can warehouse (e.g. store) information such as position or motion data, or user data, or other data associated with implementations of the present technology. Though databases 250 and 260A-C are displayed logically as single units, databases 250 and 260A-C can each be a distributed computing environment encompassing multiple computing devices, can be located within their corresponding server, or can be located at the same or at geographically disparate physical locations.

The network 220 can be a local area network (LAN) or a wide area network (WAN), but can also be other wired or wireless networks. The network 220 may be the Internet or some other public or private network. Client computing devices 210A-D can be connected to the network 220 through a network interface, such as by wired or wireless communication. While the connections between the server 230 and the servers 260A-C are shown as separate connections, these connections can be any kind of local, wide area, wired, or wireless network, including the network 220 or a separate public or private network.

Those skilled in the art will appreciate that the components illustrated in FIGS. 1 and 2 described above, and in each of the flow diagrams discussed below, may be altered in a variety of ways. For example, the order of the logic may be rearranged, substeps may be performed in parallel, illustrated logic may be omitted, other logic may be included, etc. In some implementations, one or more of the components described above can execute one or more of the processes described below.

### B. POSTURE IMPROVEMENT SYSTEMS AND METHODS

In some implementations of the present technology, a device with an application or software can be used to monitor a user's posture and provide feedback about the user's posture. Such implementations can be used as a training tool to help the user improve their posture.

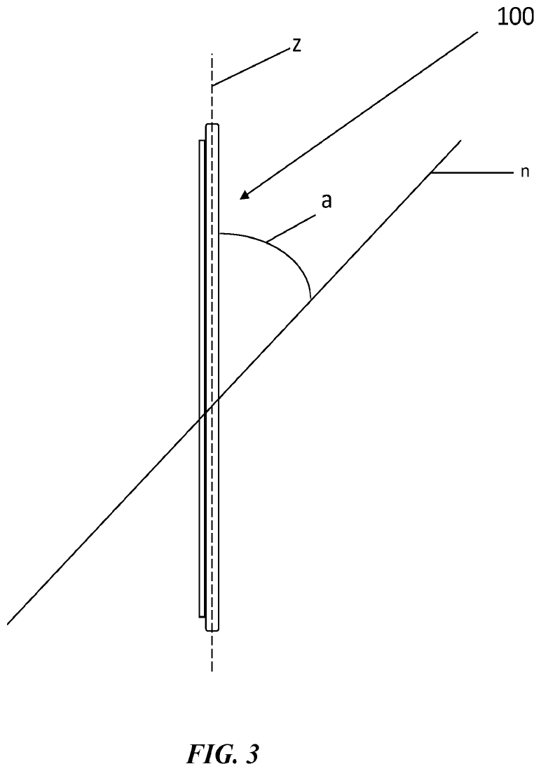

FIG. 3, for example, illustrates a schematic view of a device 100 on which some implementations can operate. The device 100 can be a smartphone, a tablet, a small computer, or another suitable small portable device. FIG. 3 shows an axis z generally representative of a vertical axis. In FIG. 3, the device 100 is generally aligned with the z-axis such that the device 100 is generally vertical. However, during use of the device 100, a user may tilt the device 100 such that it is aligned with an axis n (which is an axis that is not aligned with the z-axis). The tilt of the device 100 relative to the z-axis may be represented by expressions of values of “Z” such that: when Z=0, the device 100 is vertical and aligned with the z-axis; when Z=−1, the device 100 is horizontal and perpendicular to the z-axis; when Z=+/−0.5, the device 100 is diagonal and the angle a between the n-axis and the z-axis is approximately 45 degrees; and when Z=−0.4, the device 100 is slightly closer to horizontal than an angle a of 45 degrees.

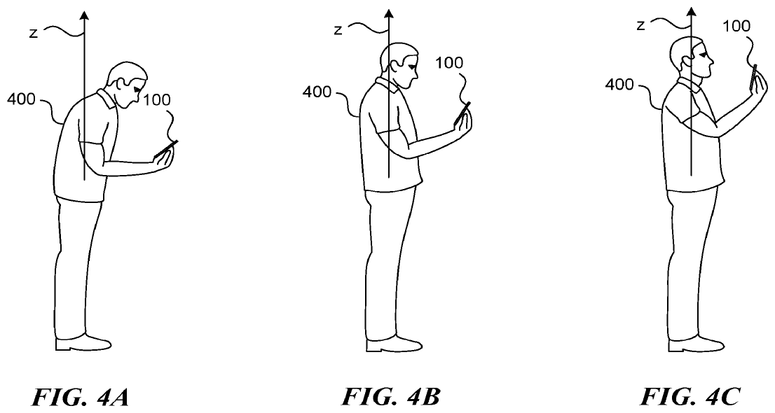

FIGS. 4A, 4B, and 4C illustrate side profile views of a user 400 engaging with the device 100. The z-axis is shown in each of FIGS. 4A, 4B, and 4C to demonstrate various postures. For example, FIG. 4A shows the user 400 in a slouched position with head tilted downward and forward toward the device 100, in a posture that may result in pain or injury. FIG. 4B shows the user 400 in a slightly improved posture, but with head positioned forward toward the device 100 and still slightly slouched. The user's 400 position in FIG. 4B may still result in pain or injury. FIG. 4C shows the user 400 in optimal or plumbline posture while operating or engaging with the device 100.

With reference to FIGS. 3, 4A, 4B, and 4C, the inventors have discovered that a near vertical or vertical angle of the device 100 (close to Z=0) correlates with healthy human posture (plumbline posture, hereinafter also referred to as “good posture”). More specifically, approximately 91% of the time, good posture correlates with holding the device 100 at Z=−0.46 to Z=0. When the device 100 was held more upright, at Z=−0.4 to Z=0, there was a correlation with good posture approximately 99% of the time. The present findings are in contrast with a common assumption that holding a device 100 flat or horizontal (close to Z=−1), always correlates with poor posture and should be corrected. However, there is a lower degree of confidence in predicting bad posture when the device 100 is farther from vertical (far from Z=0). In contrast with conventional systems that punish bad posture or provide negative feedback to users, implementations of the present technology track, analyze, and reward usage of the device 100 at angles closer to vertical (Z=0) to encourage good posture by the user.

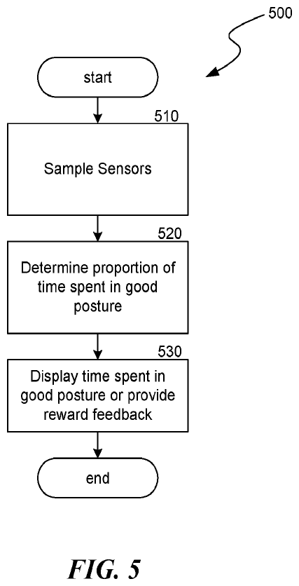

FIG. 5 is a flow diagram illustrating a process 500 used in some implementations for posture improvement systems and methods. The process 500 is illustrated as a set of operations or processes 510-530. All or a subset of the process 500 can be implemented, for example, via device 100 and features described herein with reference to FIGS. 1 and 2. Alternatively or in combination, all or a subset of the process 500 can be implemented using other suitable devices or systems.

Beginning at block 510, a controller programmed with instructions to carry out the process 500, including one or more processors 120 in the device 100 or processors connected via networking, periodically samples and causes to store in memory data from one or more I/O devices 140 associated with an orientation of the device 100 relative to the z-axis. For example, the 1/O devices 140 can include one or more accelerometers, gyroscopes, or other devices for determining orientation of the device 100. Sampling happens periodically, such as when a user is actively using an app or software containing the programmed instructions, immediately after using the app or software, or when the app or software is awoken to run in a background process. In some embodiments, sampling is only performed when a user is known to be awake and more likely to be engaging with the device 100. Sampling may be from anywhere from five seconds to ten minutes, or other sampling times. In some embodiments, sampling may be performed anytime the device 100 is unlocked or otherwise being used. In some embodiments, periodic sampling is preferable over constant sampling for several reasons. For example, constant sampling uses more power than periodic sampling, and some devices may have privacy limitations that prevent constant sampling.

At block 520, the controller determines the proportion of time the device 100 is in a position that correlates with good posture (for example, based on the Z value or angle a above). The proportion may relative to a period of time such as a day or the waking hours of a day.

At block 530, the process 500 may include providing feedback to indicate that a user has or has not complied with a good posture position (based on the device orientation) for a sufficient amount of time. For example, if a user has complied with a good posture position for a specified proportion of time, the user may be rewarded with praise. In some embodiments, for example, the controller may instruct the display 130 to show praise to reward good posture. Reward for good posture may be provided once per day, periodic during the day, or other frequencies. In some embodiments, feedback may be provided by push notifications, emails, or vibrational techniques. In some embodiments, data associated with time spent in good posture can be transmitted to a medical practitioner, coach, instructor, or other third party for further analysis. In some embodiments, the step of displaying time spent in good posture may include displaying a bar graph or other chart showing multiple users' time spent in good posture to facilitate a competition or comparison among users or between a user and averages of other users (such as a regional or global average). In some embodiments, the controller may instruct the display 130 to show a red, yellow, or green indicator corresponding to whether the user is engaging in good posture for a sufficient amount of time in a day (for example, green may indicate sufficient good posture, yellow may indicate nearly sufficient time in good posture, and red may indicate a prevalence of bad posture).

In some embodiments, the controller may receive feedback from the touchscreen and analyze the feedback to determine how often a user operates or holds a device with one hand and the controller may carry out a process similar to the process 500 to reward a user for evenly distributing time using one hand or the other.

In some embodiments, an I/O device 140 can include a GPS receiver. A controller can be further programmed with instructions to receive GPS data and analyze the data to determine if a user is moving, such as walking or driving. If a user is not walking or driving, at block 530 of the process 500, a device may reinforce (for example, with a reward or notice on the display) the positive behavior of not simultaneously walking and operating the device 100.

Advantages of implementations of the present technology include the software, process, or app being executable with a single tool (such as the device 100), without a need for external or separate devices that may risk loss or damage. Implementations of the present technology may identify plumbline posture with at least 95% accuracy, whether a user is walking, standing, or seated. The positive feedback regime takes advantage of a user's desire for positive reinforcement as opposed to negative feedback and/or punishment associated with many conventional systems.

### C. RANGE OF MOTION ANALYSIS

In some implementations of the present technology, the device 100 with an application or software can be used to monitor a user's range of motion and provide feedback about the user's range of motion. Such implementations can be used as a training tool to help the user improve their dexterity or range of motion.

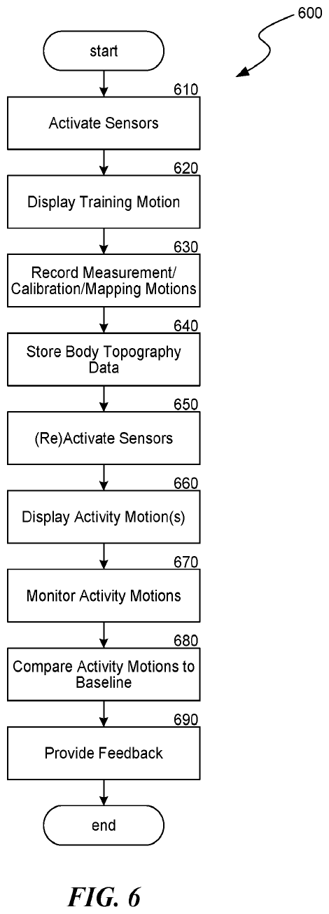

FIG. 6, for example, is a flow diagram illustrating a process 600 used in some implementations of the present technology for mapping a user's body topography, monitoring a user's motions, comparing the motions to previous motions or to an ideal motion template, and providing feedback. The process 600 is illustrated as a set of operations or processes 610-690. All or a subset of the process 600 can be implemented, for example, via device 100 and features described herein with reference to FIGS. 1 and 2. Alternatively or in combination, all or a subset of the process 600 can be implemented using other suitable devices or systems.

Process 600 begins at block 610. In some implementations, at block 610, the controller or processor can activate one or more of the sensors in the device 100, such as one or more accelerometers or gyroscopes, in response to a user opening the application or software or in response to a user engaging with a graphical user interface to request execution of the process 600. At block 620, the controller or processor causes the display to show an image or a video of a training motion the user is to perform. Representative training motions (which also may be called measurement, calibration, or mapping motions, or training exercises) are described in additional detail below. The training motions provide data regarding a user's body topography, such as limb length and relative positioning of a user's joints.

At block 630, the controller or processor records positional data associated with the training motions as the user carries out the motions. For example, one or more accelerometers or gyroscopes can provide orientation or angular velocity associated with the user's movement of the device. At block 640, the controller or processor stores the positional data as body topography data, which can be used to calibrate the size of activity motions (which may also be called evaluation exercises) as described in additional detail below. In some implementations, the process 600 may immediately proceed to the activity portion of the process 600, which begins at block 650. Alternatively, in some implementations, the process 600 may be paused to perform the activity portion at a later time.

At block 650, the controller or processor activates or reactivates the sensors. The process 600 then continues at block 660, and the controller or processor can cause the display to show proposed activity motions for a user to execute. At block 670, the controller or processor receives data from the sensor(s) as the user executes the activity motions. For example, one or more accelerometers or gyroscopes can provide orientation or angular velocity associated with the user's movement of the device. At block 680, the controller or processor can compare the data associated with the activity motions (such as a path or a series of points along a pathway) to determine how close the activity motion is to the proposed activity motion displayed at block 660. At block 690, the controller or processor can initiate a feedback algorithm to provide feedback regarding the accuracy of the activity motion. For example, the controller or processor can cause an actuator in the device 100 to vibrate or it can cause the display to show feedback information, such as a message of praise/encouragement or constructive criticism/advice based on how accurately the user executed the activity motion.

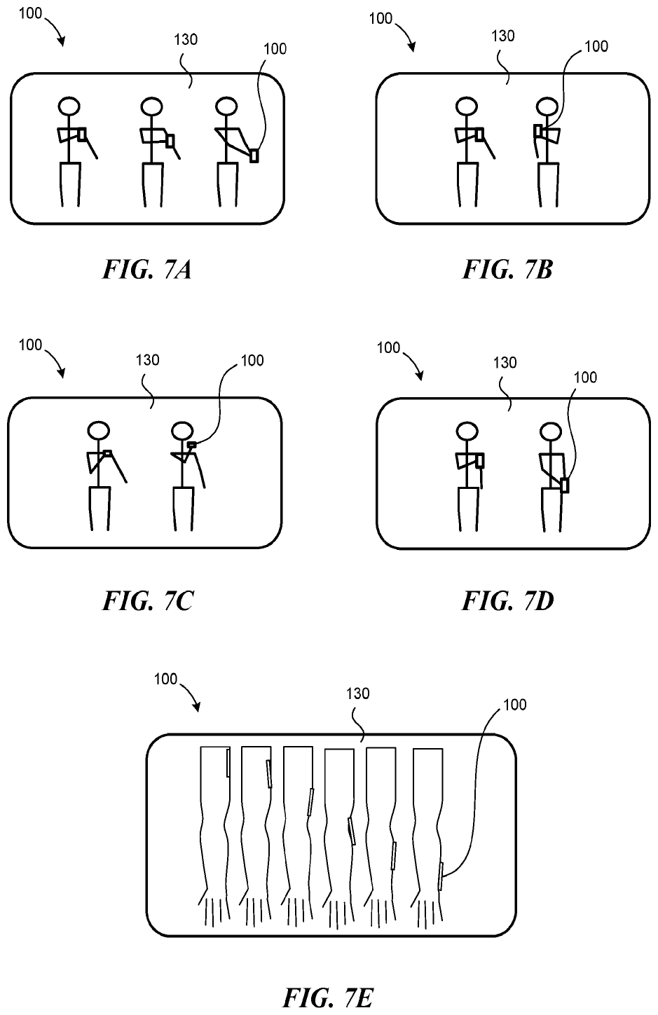

FIGS. 7A-E illustrate representative training motions in accordance with implementations of the present technology. FIG. 7A, for example, shows the device 100 with display 130, showing a sequence (from left to right) of a user moving the device 100 to perform the training motions in accordance with blocks 620 and 630 described above. In FIG. 7A, the user moves the device 100 from shoulder to elbow to wrist. At each point (shoulder, elbow, wrist), the user may press a target icon on the display 130 to indicate the device 100 is positioned at the respective point. In some implementations, the controller can invoke the device 100 to provide feedback (such as vibrational or image-based feedback) as an indicator that the icon has been pressed. In accordance with block 630 of FIG. 6, the controller or processor registers the relative position of each point, and at block 640, the controller or processor stores the positional data and extrapolates the relative distances and positions of each point. FIG. 7B is similar to FIG. 7A, but it shows a sequence of a user moving the device 100 to perform a training motion to measure the user's shoulder width. At each point (each shoulder), the user may press a target icon on the display 130 to indicate the device 100 is positioned at the respective point. FIG. 7C illustrates a training motion for measuring the length of the user's neck. FIG. 7D illustrates a training motion for measuring the length of a user's fully-extended arm.

In some implementations, a training motion may include moving the device 100 along the contour of a limb. For example, the display 130 may show an animation or sequence of images illustrating movement of the device 100 along one or both sides of a user's arm, as generally illustrated in FIG. 7E. The processor or controller instructs the display 130 to instruct a user to hold the device 100 at their shoulder with their arm (or other limb) fully extended, then draw a straight line to their wrist, keeping the phone pressed against the skin at all times. The position of the device 100 will be detected using the sensors to inform to learn the contour of the limb.

In some implementations, the controller or processor may instruct the display 130 to show the user various pressure points or other locations on the body. The device 100 can instruct the user to hold the device 100 against pressure points or other points (which can be consistent with areas of known therapeutic benefit and with the analysis of the user's biomechanical properties). In accordance with blocks 620 to 640 of FIG. 6 described above, the sensors can measure movement and relative positioning, and the controller or processor can cause data regarding the movement and relative positioning to be saved in memory. By measuring movement of pressure points, implementations of the present technology can analyze tissue density or strain. In some embodiments, one or more vibration units in the device 100 can apply therapeutic pressure.

Using the data representative of the topography of a user's body created at blocks 610 to 640 of the process 600 described above with reference to FIG. 6, implementations of the present technology can analyze and provide feedback regarding activity motions in accordance with blocks 650 to 690. In some implementations, for example, mapping of the user's body can be performed periodically to adjust for changes in the user's baseline physiology. In some embodiments, the controller may evaluate whether a measurement represents a physical impossibility and in response, it can prompt a user (via feedback) to measure again.

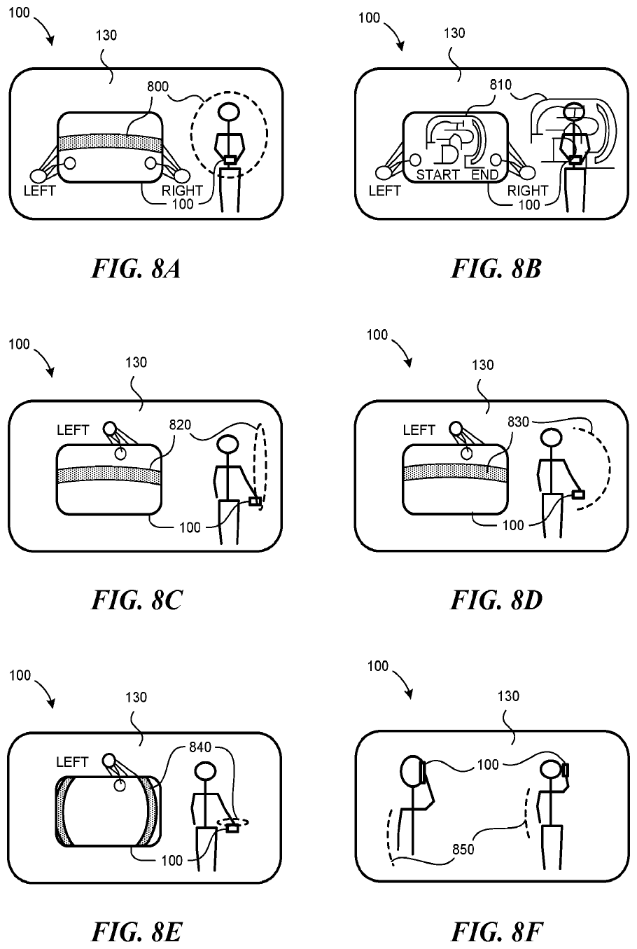

FIGS. 8A-10C illustrate example activity motions in accordance with implementations of the present technology. In each of these examples, the user may perform activity motions associated with movement of the device 100. These motions can be used in implementations of blocks 660 and 670 of the process 600 described above with reference to FIG. 6. With reference to FIGS. 8A-8F, the user is instructed to trace a shape using their device 100, while keeping other body members still. The device's accelerometer, gyroscope, speedometer, or other sensors are used to measure the speed and accuracy of tracking. Active feedback in the form of vibration, light flashes, and other case-specific methods with varying intensities, cadences and/or rhythms may be further used to mediate the user's exercise, including both corrective feedback, reinforcement feedback and time-keeping. Feedback is described in additional detail below. These motions represent specific movements that have therapeutic effects on musculoskeletal health. The user is guided using the information on the display 130. The controller or processor generates images on the display representative of virtual shapes to be traced out by the movement of the user, with the size of the virtual shape consistent with the analysis of the user's body and health needs (for example, a user's physiology, body topography, range of motion, or other aspects) performed at block 640 in FIG. 6.

FIG. 8A, for example, shows the device 100 with display 130 showing a sequence shape 800 for a user to follow to perform the activity motion. The display 130 instructs the user to hold their device 100 parallel with their frontal plane with their fingers placed as indicated and move their device 100 in a wide circular pattern, portions of which can be visible on the display 130. As the user follows the prescribed path, the display 130 may not provide visual feedback (to prevent the user from needing to look at device 100 to complete the exercise, as well as to promote natural movement. Vibrational feedback can be provided as described below. Although a circle is shown as the sequence shape 800, in various implementations other shapes can be used, e.g., a triangle, a square, a rectangle, or other shapes. For example, as generally illustrated in FIG. 8B, the shape may be in the form of an asymmetrical or random pattern or maze 810. The movements shown in FIGS. 8A and 8B can generally include circumduction movements, which can involve a combination of flexion, extension, abduction, and adduction of a limb).

FIG. 8C shows the device 100 with a sequence shape 820 for the user to follow to perform an activity motion corresponding to sagittal plane flexion and extension. The display 130 instructs the user to hold their device 100 at their left or right side, parallel with their frontal plane, and to move their device 100 in a wide circular pattern according to the shape 820.

FIG. 8D shows a sequence shape 830 for the user to follow to perform an activity motion corresponding to shoulder abduction and adduction. In this example, the device 100 instructs the user to hold their device 100 at their left or right side, parallel with their frontal plane, and to move their device 100 in a wide semi-circular pattern, which engages the shoulder in an abduction and adduction pattern.

FIG. 8E shows a sequence shape 840 for the user to follow to perform an activity motion corresponding to shoulder circumduction. The device 100 instructs the user to hold their device 100 at their side, parallel with their frontal plane and to move their smartphone in a narrow semicircular path, which engages the shoulder in circumduction.

FIG. 8F shows a sequence shape 850 for the user to follow to perform an activity motion corresponding to shoulder flexion and extension. The device 100 instructs the user to hold their device (e.g., smartphone) against their cheek as though attending to a telephone call, and to bend their torso forward and backward, keeping their device 100 still relative to their face, which engages the back and vertebral column in flexion and extension.

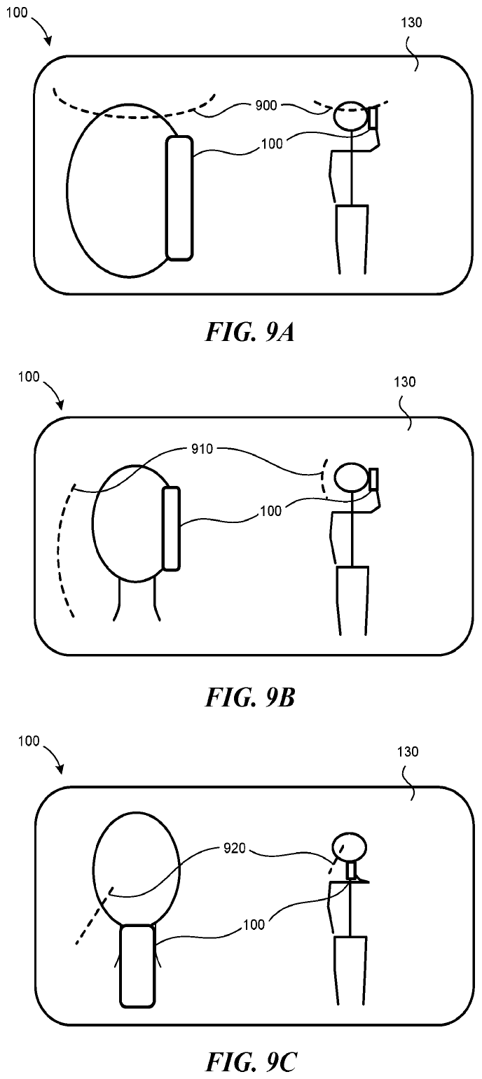

FIGS. 9A-9C illustrate additional example activity motions that can be used in implementations of blocks 660 and 670 of the process 600 described above with reference to FIG. 6. Implementation of these motions is generally similar to implementation of the motions described above with reference to FIGS. 8A-8F, in that a user traces a shape with the device 100 suitably positioned, but the motions of FIGS. 9A-9C focus on engagement of the user's neck and head. FIG. 9A, for example, shows a sequence shape 900 for a user to follow to perform an activity motion corresponding to neck rotation. The device 100 instructs the user to hold their device 100 against their cheek as though attending a phone call, and to move their head while keeping their device 100 still relative to their face, which engages the neck in rotation. FIG. 9B shows a sequence shape 910 for the user to follow to perform an activity motion corresponding to neck flexion and extension. The device 100 instructs the user to hold their device 100 against their cheek as though attending a phone call and to move their head downward and upward, keeping their phone still relative to their face, which engages the neck in flexion and extension.

FIG. 9C shows a sequence shape 920 for the user to follow to perform an activity motion corresponding to mandible retraction and protraction. In particular, the device 100 instructs the user to hold the device 100 against their chin and move their jaw forward and backward, keeping their device 100 still relative to their face, which engages the mandible in retraction and protraction.

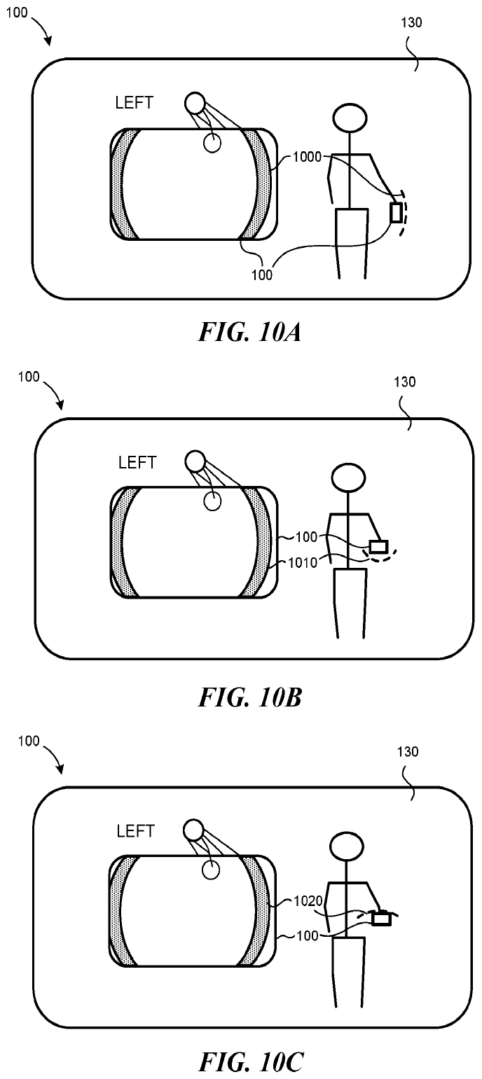

FIGS. 10A-10C illustrate further additional activity motions in which a user traces a shape with the device 100, with a focus on wrist movement. For example, FIG. 10A illustrates the device 100 with display 130 instructing a user to hold the device 100 and move the device 100 with their wrist along a sequence shape 1000. The device 100 instructs the user to hold their device 100 at their left or right side, parallel with their frontal plane, and to rotate their wrist upward and downward, which engages the wrist in flexion and extension. The device 100 can instruct the user to hold the device 100 in a manner that positions the wrist facing upward or downward. The device 100 can instruct the user to hold the device 100 differently, such as in a position that faces the wrist upward or downward, or in a position in which the forearm is transverse to the parallel plane. The device 100 can instruct the user to move the device left to right to engage the wrist in pronation and supination.

FIG. 10B shows the device 100 instructing the user to hold their device 100 flat, keeping their forearm parallel with the transverse plane, while rotating the wrist clockwise and counterclockwise along sequence shape 1010, which will engage the wrist in pronation and supination. FIG. 10C shows the device 100 instructing the user to hold their device 100 flat, keeping their forearm parallel with the transverse plane, while rotating their wrist to the left and right along sequence shape 1020, which will engage their wrist in pronation and supination.

While FIGS. 7A-10C described above relate to movements of the device 100 in predetermined activity motions or training motions, in some implementations, a user may perform activity motions or training motions on a stable or fixed device 100 with the user navigating their fingers on the display 130.

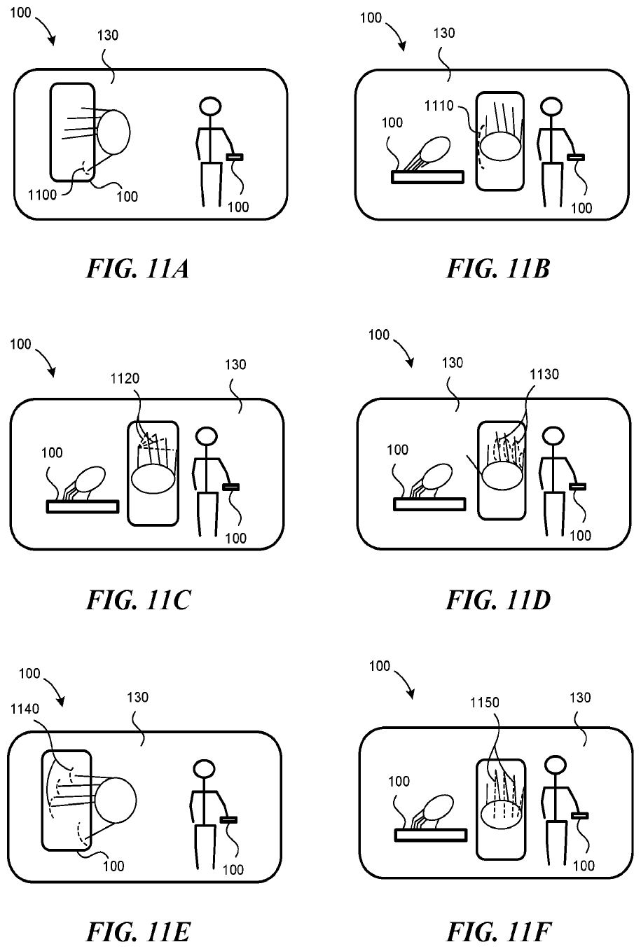

FIGS. 11A-11F, for example, illustrate such thumb and finger activity motions. In some implementations, the app or software may include the controller or processor displaying calibration or mapping motions on the display to set a baseline for a user's motion, however, in further implementations, the app or software may include pre-set optimal motion paths for comparing against a user's activity motion. Referring first to FIG. 11A, the display 130 of the device 100 instructs the user to place their fingers against the display 130. The controller or processor can cause the display 130 or a vibration unit within the device 100 to provide guidance as the user moves their thumb along sequence shape 1100 to engage in flexion and extension.

FIG. 11B illustrates the device 100 with display 130 instructing the user to place their fingers against the display 130. As noted previously, the controller or processor can cause the display 130 or a vibration unit within the device 100 to provide guidance as the user moves their thumb along sequence shape 1110 to engage the thumb in adduction and abduction. In some implementations, as generally illustrated in FIG. 11C, the controller or processor can cause the display 130 or a vibration unit within the device 100 to provide guidance as the user moves their thumb to engage in opposition with each finger, along sequence shapes 1120 (the thumb is to move to each opposing finger).

In some implementations, as generally illustrated in FIG. 11D, the controller or processor can cause the display 130 or a vibration unit within the device 100 to provide guidance as the user moves their fingers along sequence shapes 1130 (simultaneously or individually opening and closing fingers) to engage in a tendon stretching motion. In some implementations, as generally illustrated in FIG. 11E, the display 130 instructs the user to move their fingers along the display 130 side to side along sequence shapes 1140. In some implementations, as generally illustrated in FIG. 11F, the display 130 instructs the user to move their fingers along the display 130 in abduction and adduction (curling the fingers) along sequence shapes 1150.

In each activity motion described above with reference to FIGS. 8A-11F, visual or vibrational feedback can guide the user. In other implementations, however, no guidance is provided while the user completes the activity motions.

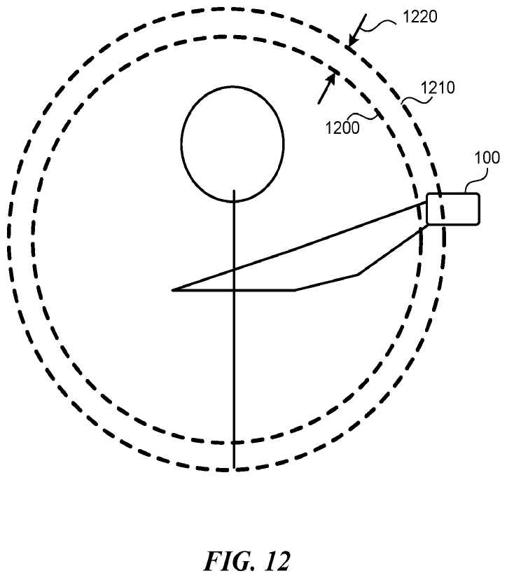

In some embodiments, the controller or processor facilitates provision of feedback on the basis of a user's deviation from an ideal or pre-set motion path. Deviation can be analyzed in real time and after completion of an activity motion. FIG. 12, for example, illustrates a user's deviation in terms of a distance from the ideal or pre-set motion path. In this example, an ideal or pre-set motion path 1200 may have a set size or diameter, but the user may move the device 100 along a real path 1210. The feedback process includes measuring a deviation 1230 between the pre-set path 1200 and the real path 1210. In some implementations, feedback may be provided in real time based on the deviation 1230 from the pre-set or ideal path. The process can include the controller or processor measuring deviation during the activity motion and causing the display 130 to show an alert symbol or message or causing a vibration unit to vibrate to alert the user to the deviation. In some implementations, a user's speed may be tracked during the motion. If the user's speed in executing the activity motion deviates from an ideal or pre-set speed, the controller or processor can cause the display or vibration unit to alert the user. In some implementations, intensity of the vibration may correlate to the magnitude of the deviation (for example, if the controller or processor determines relatively large deviation, the vibration intensity may be increased, or if the controller or processor determines relatively small deviation, the vibration intensity may be decreased or eliminated). In some embodiments, deviation can be based on the displacement of a center point of the device 100 or another reference point in the device from the pre-set or ideal path.

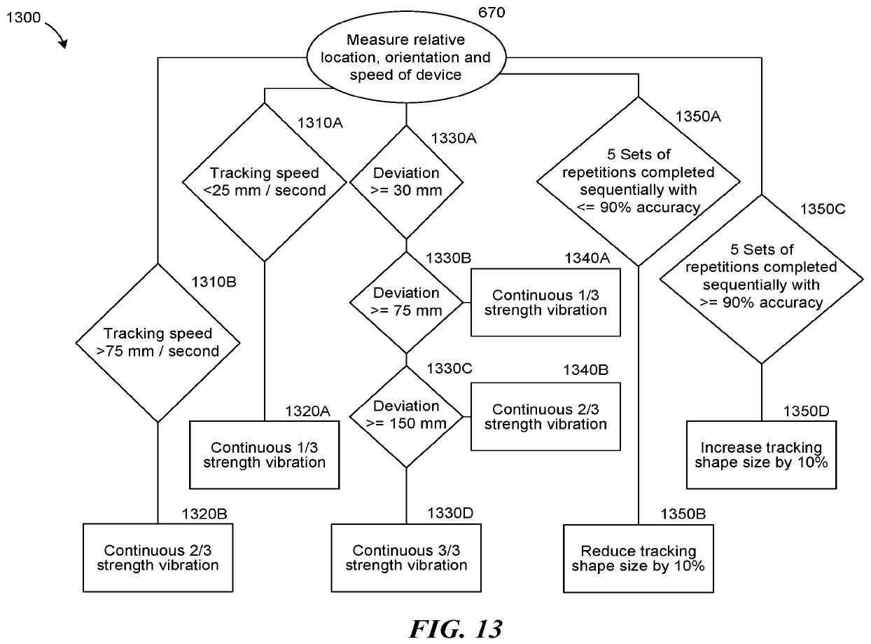

FIG. 13 is a flow diagram illustrating a process 1300 used in some implementations of the present technology for monitoring and providing feedback about activity motions. The process 1300 may be utilized, for example, in conjunction with blocks 670 to 690 of the process 600 described above with reference to FIG. 6. The process 1300 is illustrated as a set of operations or processes 1310-1350. In some implementations, all or a subset of the process 1300 may be executed by the controller or processor to provide haptic (vibration) feedback.

In some implementations, the process 1300 may be executed to determine the size of the sequence shape performed by a user during an activity motion. For example, if a user successfully completes an activity motion a number of times, the process 1300 can increase the size of the sequence shape to further challenge the user or as a reward for improved range of motion. If a user completes an activity motion a number of times unsuccessfully (for example, below an acceptable threshold of accuracy), then the process 1300 can decrease the size of the sequence shape.

Referring to FIG. 13, at block 670, the controller or processor receives data from the sensors as the user executes the activity motions. The process 1300 continues at blocks 1310A and 1310B, and then at blocks 1320A and 1320B, in which the speed of the movement of the device is compared to a threshold and, depending on that comparison, the controller or processor causes the device to perform a vibration. For example, in some implementations of process 1300, at block 1310A, if the speed of the device is less than 25 millimeters per second, a continuous vibration of ⅓ strength can be performed to indicate to the user that they should speed up (block 1320A). At block 1310B, if the speed of the device is greater than 75 millimeters per second, then at block 1320B, a continuous vibration of ⅔ strength can be performed to indicate to the user that they should slow down.

In some implementations, the process 1300 can analyze deviation. At block 1330A, for example, if the deviation is less than thirty millimeters, no vibration may be performed as the user is within an acceptable range. However, if the deviation is greater than or equal to 30 millimeters, but is less than 75 millimeters, at blocks 1330B and 1340A, a ⅓ strength vibration may occur to warm the user. At blocks 1330C and 1340B, if the deviation is greater than or equal to 75 millimeters but is less than 150 millimeters, a ⅔ strength vibration may be invoked. At block 1330D, if the deviation is greater than or equal to 150 millimeters, a 3/3 (full) strength vibration may be invoked.

In some implementations, the process 1300 can analyze the number of successful activity motions carried out. For example, at blocks 1350A and 1350B, if five sets are completed unsuccessfully (which may mean less than or equal to an accuracy of 90% of the pre-programmed ideal speed or deviation), the sequence or tracking shape size may be reduced by an amount such as ten percent, to make the activity easier for a user or to accommodate a smaller range of motion. If five sets are completed successfully, however, at blocks 1350C and 1350D, the sequence or tracking shape size may be increased by an amount such as ten percent, to make the activity more challenging or to accommodate a larger range of motion. The process 1300 can be implemented for activity motions in which the user moves the phone. It will be appreciated that the specific ranges/dimensions provided in FIG. 13 are associated with specific embodiments of the present technology, and one or more of these ranges/dimensions may be changed in additional embodiments of the present technology.

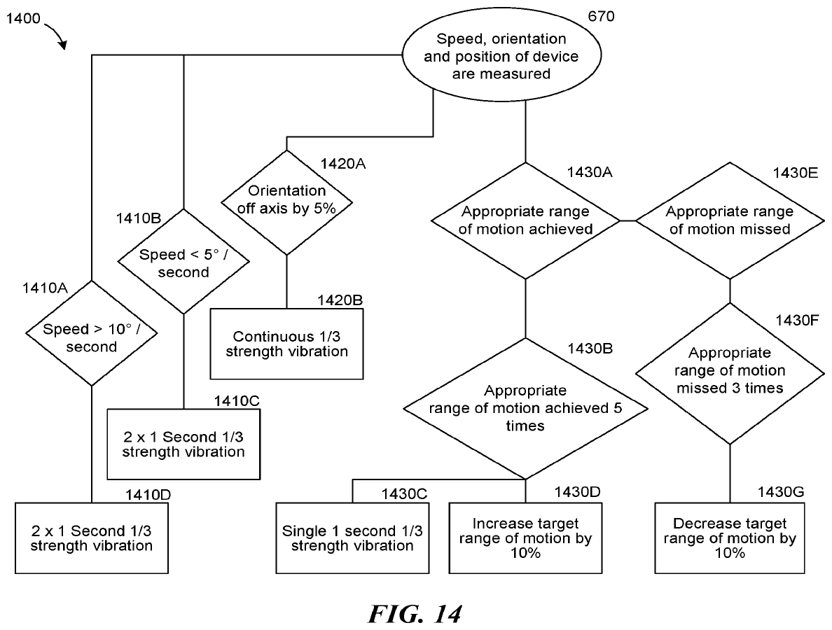

FIG. 14 illustrates a process 1400 for analyzing activity motions with a focus on wrist movement, such as the activity motions described above with regard to FIGS. 10A-10C. As with the process 1300 described above, the process 1400 may be utilized in conjunction with block 670 of the process 600 of FIG. 6. Referring first to blocks 1410A and 1410B, if the speed of the movement of the device is greater than 10 degrees per second or less than five degrees per second, then at blocks 1410C and 1410D, the controller can invoke a vibration sequence, such as two one-second vibrations at ⅓ strength to alert the user that they are performing the motion too fast or too slow. At block 1420A, the controller can determine if the device orientation drifts off of a pre-set axis (such as an axis about which the activity motion is to be performed, for example, a rotation axis about which the device is to be rotated or pivoted) by 5% or more, then at block 1420B, a continuous ⅓ strength vibration can be induced by the controller until the user corrects the deviation. At block 1430A, the controller determines if the appropriate range of motion as defined by the software is reached at least one time in either direction. If the appropriate range is reached, the user can continue the movement. At block 1430B, the user continues the movement until a specified number of motions (such as 5 repetitions of the motion) is reached. When that specified number is reached, at block 1430C, the controller can invoke a short (such as one second) ⅓ strength vibration, and at block 1430D, the range of motion can be increased (for example, by 10%). At blocks 1430E and 1430F, if, during the course of motion, the appropriate range of motion as defined by the software is not met three times, then the range of motion can be decreased at block 1430G (for example, by 10%). In some implementations, all vibrations and changes to range of motion targets can be displayed on the display of the device with a visual cue or provided in audio feedback. As noted, the specific ranges/dimensions provided in FIG. 14 are associated with specific embodiments of the present technology, and one or more of these ranges/dimensions may be changed in further embodiments of the present technology.

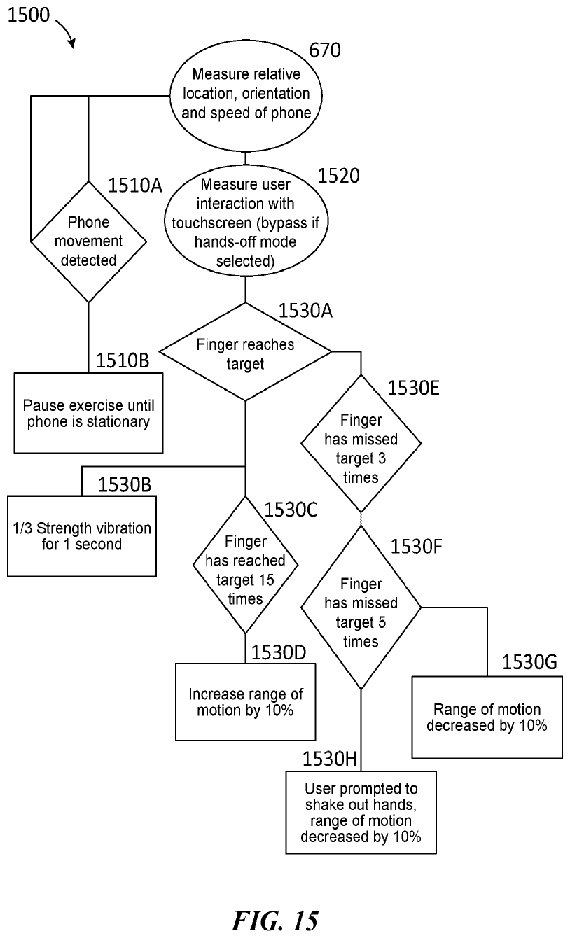

FIG. 15 illustrates a process 1500 for analyzing activity motions on a stable or fixed device with the user navigating their fingers on the display (for example, activity motions described above with regard to FIGS. 11A-11F). The process 1500 may be utilized, for example, in conjunction with block 670 of the process 600 of FIG. 6. Beginning at block 1510A, the controller or processor analyzes whether the device is moving at all. If the device is moving less than a suitable threshold, then the activity motions can proceed. If the device is moving more than a suitable threshold, then at block 1510B, the device pauses the activity until the device is stable again. Optionally, the display may show visual feedback to warn the user to hold the device stable. At block 1520, the controller or processor analyzes the user's interaction with the input device or touchscreen. At block 1530A, if the user's device determines that the user's finger has reached the target or adequately performed that activity motion, at block 1530B, the controller may invoke a ⅓ strength vibration for 1 second, or another strength of vibration for a different amount of time, to notify the user of completion of a single repetition in a set. At block 1530C, if the user's finger reaches the target a number of times, such as fifteen times, the controller may change the target position (for example, increase the range of motion by ten percent) at block 1530D. At block 1530E, the controller determines whether the user's finger missed the target a number of times (for example, three times). If the user has not missed the target three or more times, the process 1500 continues and the user carries out the activity motion again and again. At blocks 1530F and 1530G, if the user has missed the target at least three times, but less than five times, the controller or processor can decrease the range of motion of the activity by an amount, such as ten percent. However, at block 1530H, if the user has missed the target at least five times, the controller may prompt the display to instruct the user to shake their hands or relax in addition to decreasing the range of motion of the activity. The specific ranges/values provided in FIG. 15 are associated with specific embodiments of the present technology, and one or more of these ranges/values may be changed in additional embodiments of the present technology.

In some implementations, if a user is unable to make consistent, comfortable contact with the display or touchscreen (for example, because the user has long nails or another obstruction), then the user can select a non-contact motion option (a bypass mode), allowing the user to track the visual guide on the display rather than interacting directly with the guide and experiencing the haptic range associated with the motion.

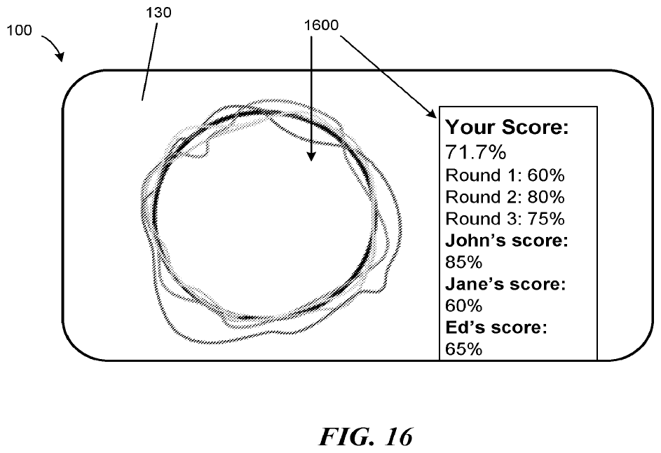

FIG. 16 illustrates the device 100 with display 130 showing graphical feedback 1600 in accordance with some implementations of the technology. In some implementations, the user's performance in an activity motion can be quantified both numerically as a percentage of the virtual shape tracked accurately, as well as graphically as a space of their attempts at achieving the prescribed shape. In some implementations, tracking results from each of the number of selected rounds or repetitions of the movement is displayed. For example, FIG. 16 shows three rounds, each of which may be color-coordinated or otherwise identifiable relative to the graphic representation of the activity motion. The scores may be based on accuracy in speed, deviation, or other factors described above. Although distances of deviation, speed, and other accuracy thresholds are described above, these are examples only, and in other implementations, other metrics may be used.

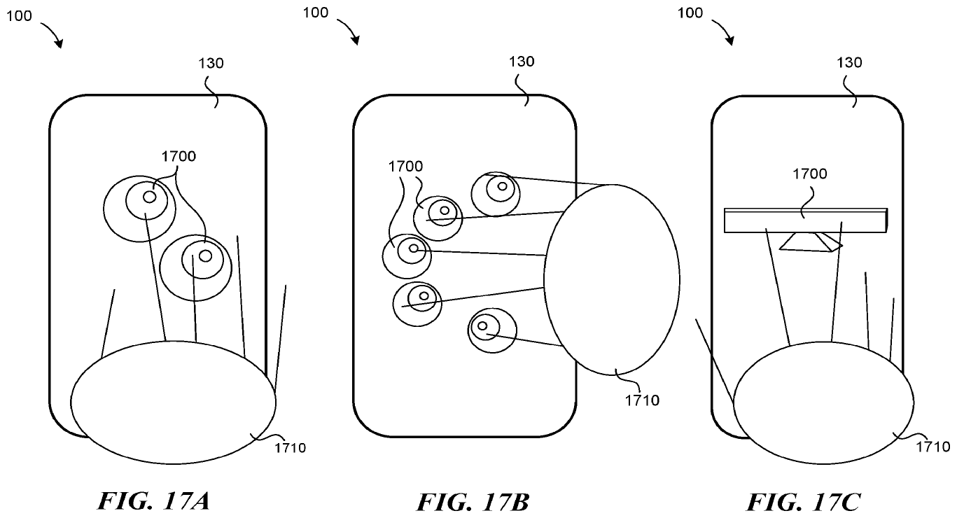

FIGS. 17A, 17B, and 17C illustrate additional activity motions that may be accomplished on a stationary device, in a manner similar to the motions described above with regard to FIGS. 11A-11F. However, in some implementations, activity motions can include interaction with a three-dimensional or simulated three-dimensional virtual environment or object 1700 configured to be displayed on the two-dimensional device display 130. For example, in some implementations, the activity motions can include a user using their fingers or hands 1710 to interact with 3D spheres (FIG. 17A), moving touchpoints that a user must follow to achieve high accuracy scores (FIG. 17B), or a balancing beam (FIG. 17C).

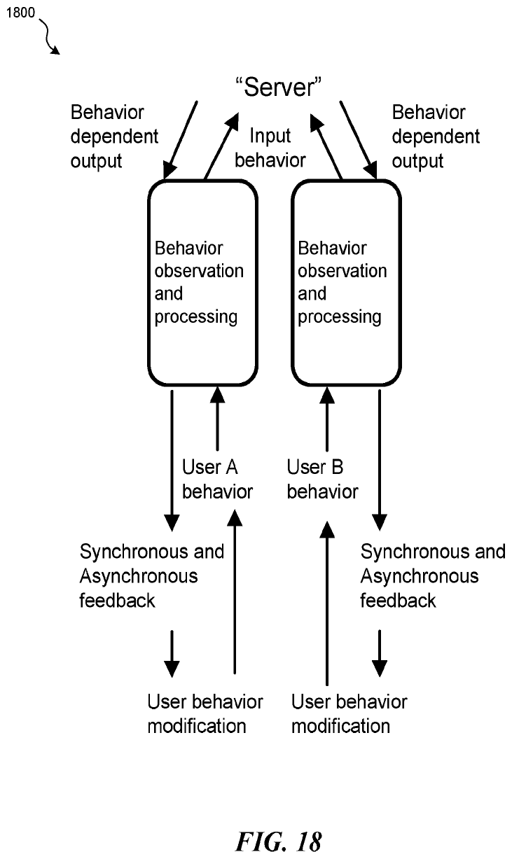

FIG. 18 illustrates a schematic diagram 1800 of various implementations of the present technology. In some implementations, for example, the device 100 may act as a sensor hub by collecting and processing relevant data such as the unique speed, position, and orientation data (for example, using the device's accelerometers or gyroscopes) of a user's device. In some implementations, the device 100 and the programmed instructions thereon can be used to correlate and review data for statistical deviation to quantify performance. In some implementations, however, in which multiple users may perform an activity motion concurrently, each of their two devices acts as a sensor hub, wherein data is processed from each device and analyzed by the controller or processor within the device. The data and the processed outputs can be further sent to and analyzed by a central server which can transmit behavior-dependent output information back to the devices 100 to provide feedback. In some implementations, there may be more than two devices (for example, more than two users), and each of those devices can connect to one or more servers for sending data and receiving feedback.

Although motion of a user's device 100 or motion of a user's fingers relative to a device 100 are described above, in some implementations, the jitter or shake of a device 100 can be analyzed. For example, the absence of jitter, which can correlate to an absence of underlying tremors of the user, can be periodically sampled and rewarded in a manner similar to good posture described above with regard to FIG. 5. Intermittent sensing via the device's accelerometer or gyroscope allow for the measurement of jitter while the user holds the device relatively steady in in one position and orientation. Study of jitter can allow for the analysis of a user's hydration level or mental state, as diminished hand motor control can indicate dehydration or a stressed mental state. Jitter data can be used to inform visual feedback, expressed through positive feedback to encourage behaviors to minimize tremor incidence, including meditation and hydration.

Implementations of the present technology can include user grouping and group reinforcement. For example, in some implementations, one or more users may be linked to a group of other users (who may optionally have similar body sizes or ranges of motion). In some implementations, after one user has completed part of one task, the remainder of the task can only be completed after some interaction or task completion by another user. In some implementations, activities must be completed at certain times in order to unlock activities for other users. In some implementations, groups of users may be required to perform activities at the same time.

Although feedback is described herein as haptic (vibrational) or visual (on-screen), in various implementations, feedback can include any other suitable feedback, such as acoustic, light, or other feedback. Vibrational feedback can vary in duration, intensity, rhythm, or frequency.

In some implementations, data regarding the user's activity motions or posture can be transmitted to a remote interface to facilitate monitoring the performance and evaluation of the user. In some implementations, activity motions can include movements associated with sports or meditation techniques, such as yoga. For example, in some embodiments, a yoga teacher may calibrate or program the correct activity motion, the device can display the correct movement, and a user can attempt to replicate that movement, receiving a score in a manner similar to other feedback described above. In some implementations, various processes can include a push notification to multiple users to perform an activity at the same time with scores based on the accuracy and timeliness of the activity. In some embodiments, the device 100 may receive data regarding the training or activity motions from external sensors, such as a smartwatch or other wearable devices.

Advantages of implementations of the present technology include the software, process, or app being executable with a single tool (such as the device 100), without a need for external or separate devices that may risk loss or damage. Implementations of the present technology can be used to analyze a user's range of motion and to assist the user in increasing their range of motion by providing feedback or adjusting the activity motions in response to the analysis.

### D. ADDITIONAL EXAMPLES

Several aspects of the present technology are set forth in the following examples. The examples may be combined with each other, and further examples of the present technology include more, fewer, or different elements than the elements in the examples.

1. A method for monitoring and providing feedback regarding motion of a mobile electronic device associated with motion of a user of the device, the method comprising:
displaying, on the device, a training motion for a user to perform;
sensing, using a sensor of the device, first actual motion of the user;
storing, in a memory, data relating to topography of the user's body based on the first actual motion;
displaying, on the device, a first activity motion for a user to perform, the first activity motion being different from the training motion;
sensing, using a sensor of the device, second actual motion of the user;
storing, in the memory, data relating to the second actual motion of the user;
comparing the data relating to the second actual motion of the user to the first activity motion; and based on the comparison, providing feedback regarding accuracy of the motion.

2. The method of example 1, wherein using a sensor of the device comprises using an accelerometer or a gyroscope.

3. The method of example 1 or 2, wherein providing feedback comprises causing a display of the device to show a graphical representation of accuracy of the motion.

4. The method of example 3, wherein causing the display to show a graphical representation of performance comprises causing the display to show a score based on the comparison.

5. The method of any one of examples 1-4, wherein providing feedback comprises causing a speaker of the device to output a noise or causing a vibration unit to vibrate.

6. The method of any one of examples 1-5, further comprising displaying, on the device, a second activity motion for a user to perform, the second activity motion being different from the first activity motion and the training motion, wherein the second activity motion is based at least in part on the comparison of the data relating to the first actual motion of the user to the second actual motion.

7. A method for monitoring and providing feedback regarding motion of a user of a portable electronic device, the method comprising:

displaying, on the device, an activity motion for a user to perform;

sensing, via the device, the actual motion of the user;

storing, in a memory, data relating to the actual motion of the user;

comparing the data relating to the actual motion of the user to the activity motion; and

based on the comparison, providing feedback regarding accuracy of the actual motion.

8. The method of example 7, wherein displaying the activity motion comprises displaying a target for a user's finger to reach or a movement for a user's finger to perform.

9. The method of example 7 or 8 wherein the activity motion comprises a first activity motion, and wherein the method further comprises displaying a second activity motion that comprises a larger or smaller range of motion based on the comparison.

10. The method of example 9, wherein displaying the second activity motion comprises displaying a smaller range of motion if the comparison corresponds to the user missing the target or failing to perform the movement.

11. A computer-readable storage medium storing instructions that, when executed, cause a mobile electronic device to perform a process for monitoring and providing feedback regarding motion of the device associated with motion of a user of the device, the process comprising:
displaying, on the device, a training motion for a user to perform;
sensing, using a sensor of the device, first actual motion of the user based on a user's motion of the device;
storing, in a memory, data relating to topography of the user's body based on the first actual motion;
displaying, on the device, an activity motion for a user to perform, the activity motion being different from the training motion;
sensing, using the sensor of the device, second actual motion of the user;
storing, in the memory, data relating to the second actual motion of the user;
comparing the data relating to the second actual motion of the user to the activity motion; and
based on the comparison, providing feedback regarding accuracy of the actual motion.

12. The computer-readable storage medium of example 11, wherein sensing second actual motion of the user comprises using an accelerometer or a gyroscope.

13. The computer-readable medium of example 11 or 12, wherein providing feedback comprises causing a display of the device to show a graphical representation of accuracy of the motion.

14. A method for monitoring and providing feedback regarding posture of a user of a portable electronic device, the method comprising:
periodically sampling a sensor in a device to determine the orientation of the device;
determining a proportion of time the device is in an orientation in which the device is oriented at an angle between 70 and 100 degrees relative to horizontal; and
periodically providing feedback regarding the proportion of time, wherein providing feedback comprises displaying, on a display of the device, information related to the proportion of time.

15. The method of example 14, wherein periodically sampling a sensor comprises periodically receiving data, in a processor or memory, from an accelerometer or a gyroscope.

16. A system for monitoring and providing feedback regarding motion of a user of a device, the system comprising:
one or more processors; and
one or more memories storing instructions that, when executed by the one or more processors, cause the computing system to perform a process comprising:
   displaying, via a display of the device, a training motion for a user to perform;
   sensing, via one or more sensors of the device, a first actual motion of the user;
   storing first data representative of the first actual motion;
   displaying, via the display, an activity motion for a user to perform, wherein the activity motion is based at least in part on the first actual motion;
   sensing, via the one more sensors of the device, a second actual motion of the user;
   storing second data representative of the second actual motion;
   comparing the first data to the second data; and
   based on the comparison, providing feedback regarding accuracy of the second actual motion.

17. The system of example 16, wherein the activity motion comprises a first activity motion, and wherein the instructions further comprise displaying, via the display, a second activity motion that comprises a larger or smaller range of motion based on the comparison.

18. The system of example 17, wherein displaying the second activity motion comprises displaying a smaller range of motion if the comparison corresponds to the accuracy of the second actual motion being below a selected value.

19. The system of any one of examples 16-18, wherein the one or more sensors comprises an accelerometer or a gyroscope.

20. The system of any one of examples 16-19, wherein providing feedback comprises displaying graphical information on the display or providing an audio signal output from a speaker of the device.

21. A testing and training system to reduce neck and back pain associated with using mobile electronic devices by improving a user's body position and movement, the system comprising:
an application operating on a mobile electronic device, the application including:
   a body position evaluation subsystem for evaluating the posture of a user utilizing data collected when the user is using or interacting with the mobile electronic device, the data including data received from one or more sensors, the body position evaluation subsystem configured to evaluate the data and to provide a body position evaluation for the user;
   an expert subsystem for prescribing an exercise plan for the user based on the body position evaluation provided by the body position evaluation subsystem, the exercise plan comprising one or more exercises or stretches specifically tailored for relieving neck and back strain of the user; and
   a training subsystem for administering the prescribed exercise plan received from the expert subsystem to the user, the prescribed exercise plan administered as an interactive exercise plan including one or more body position exercises for the user, wherein the training subsystem is designed to both deliver instruction through the mobile electronic device on proper execution of the exercises as well as to collect performance data for the user while the user is performing the one or more body position training exercises and provide feedback to the subject;
   wherein the expert subsystem is adapted to modify the interactive exercise plan as the user progresses in accordance with a level of performance of the user based on evaluating the performance data for the user provided by the training subsystem,
   wherein updates to the interactive exercise plan are determined based on at least one of the body position evaluation provided by the body position evaluation subsystem, the performance data provided by the training subsystem, and input from the user, and
wherein the application enables the user using the mobile electronic device to evaluate body position and to receive one or more exercise plans tailored for improving body position of the user to reduce stress on the neck and back.

22. The system of example 21, further comprising a remote interface configured to permit monitoring the performance of the user and controlling the evaluation and training of the user.

23. The system of example 21, wherein one of the sensors is a camera.

24. The system of example 21, wherein one of the sensors is a microphone.

25. The system of example 21, wherein one of the sensors is a tactile feature of the mobile electronic device.

26. The system of example 21, wherein a display of the mobile electronic device is used to provide visual representation of proper performance of the exercises.

27. The system of example 21, wherein a speaker in the mobile electronic device is used to provide instruction and feedback on the proper performance of the exercises.

28. The system of example 21 in which periodic feedback on body position and movement is provided to the user

29. The system of example 28 wherein the feedback is provided via graphical representation of performance.

30. The system of example 21, further comprising a remote subsystem on a remote server in communication with the mobile electronic device, the remote subsystem being configured to receive data from the mobile electronic device and send software updates to the mobile electronic device for at least one of the evaluation subsystem, the expert subsystem and the training subsystem.

31. The system of example 21, further comprising an incentive subsystem for providing motivational messages to the user on the display of the mobile electronic device.

32. The system of example 21, where an algorithm in the application is used to periodically sample data from the sensors, as opposed to continuous sampling.

33. The system of example 21, wherein the mobile electronic device is adapted to be held in the hand by the user during performance of one or more of an evaluation exercise and a training exercise.

34. The system of example 33, wherein the mobile electronic device is held by hand against a specified area of the body of the user during performance of one or more of an evaluation exercise and a training exercise.

35. The system of example 21, wherein the mobile electronic device is adapted to be attached to the user during performance of one or more of an evaluation exercise and a training exercise.

36. The system of example 21, further comprising, before evaluating body position and motion, collecting information from the user regarding self-reported age, physical activity level, level of confidence regarding ability to perform physical activity, hydration habits, mood, and understanding and familiarity of posture-related health risks.

37. The system of example 36 wherein sensors in the mobile electronic device are configured to obtain physiology data about the user.

38. The system of example 37 wherein the expert subsystem uses the data to customize exercises for the user.

39. The system of example 21 wherein the data from the sensors in the mobile electronic device are complemented by data from other external sensors, such as wearables like a smartwatch, that transmit data to the mobile electronic device or remote server to be used by the expert subsystem to evaluate body position and motion.

40. A method of testing and training body positioning and movement to reduce neck and back pain associated with the use of mobile electronic devices, comprising use of the system in any one of examples 21-39.

### E. CONCLUSION

The computing devices on which the described technology may be implemented can include one or more central processing units, memory, input devices (e.g., keyboard and pointing devices), output devices (e.g., display devices), storage devices (e.g., disk drives), and network devices (e.g., network interfaces). The memory and storage devices are computer-readable storage media that can store instructions that implement at least portions of the described technology. In addition, the data structures and message structures can be stored or transmitted via a data transmission medium, such as a signal on a communications link. Various communications links can be used, such as the Internet, a local area network, a wide area network, or a point-to-point dial-up connection. Thus, computer-readable media can comprise computer-readable storage media (e.g., “non-transitory” media) and computer-readable transmission media.

Reference in this specification to “implementations” (e.g. “some implementations,” “various implementations,” “one implementation,” “an implementation,” etc.) means that a particular feature, structure, or characteristic described in connection with the implementation is included in at least one implementation of the disclosure. The appearances of these phrases in various places in the specification are not necessarily all referring to the same implementation, nor are separate or alternative implementations mutually exclusive of other implementations. Moreover, various features are described which may be exhibited by some implementations and not by others. Similarly, various requirements are described which may be requirements for some implementations but not for other implementations.

As used herein, being above a threshold means that a value for an item under comparison is above a specified other value, that an item under comparison is among a certain specified number of items with the largest value, or that an item under comparison has a value within a specified top percentage value. As used herein, being below a threshold means that a value for an item under comparison is below a specified other value, that an item under comparison is among a certain specified number of items with the smallest value, or that an item under comparison has a value within a specified bottom percentage value. As used herein, being within a threshold means that a value for an item under comparison is between two specified other values, that an item under comparison is among a middle specified number of items, or that an item under comparison has a value within a middle specified percentage range. Relative terms, such as high or unimportant, when not otherwise defined, can be understood as assigning a value and determining how that value compares to an established threshold. For example, the phrase “selecting a fast connection” can be understood to mean selecting a connection that has a value assigned corresponding to its connection speed that is above a threshold.

As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc.

Although the subject matter has been described in language specific to structural features or methodological acts, it is to be understood that the subject matter defined in the appended claims is not necessarily limited to the specific features or acts described above. Specific embodiments and implementations have been described herein for purposes of illustration, but various modifications can be made without deviating from the scope of the embodiments and implementations. The specific features and acts described above are disclosed as example forms of implementing the claims that follow. Accordingly, the embodiments and implementations are not limited except as by the appended claims.

Any patents, patent applications, and other references noted above are incorporated herein by reference. Aspects can be modified, if necessary, to employ the systems, functions, and concepts of the various references described above to provide yet further implementations. If statements or subject matter in a document incorporated by reference conflicts with statements or subject matter of this application, then this application shall control.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Moreover, the various embodiments described herein may also be combined to provide further embodiments. For example, in some embodiments, a device can include a microphone or a camera, and the microphone or camera can facilitate training motions and monitoring and evaluation of activity motions. In some embodiments, a device can include a speaker or other audio device, and the speaker or other audio device can output audible instructions in addition to, or alternative to, instructions displayed on screen. Although upper limbs such as arms and hands are described and illustrated, other limbs such as legs or feet may be analyzed in other implementations. Although in some embodiments, a user can hold the device while performing training or activity motions, however, in further embodiments, the device can be attached to the user with a strap or other suitable attachment system. In some embodiments, the device, processor, or controller can request (via the display) information from a user regarding age, physical activity level, level of confidence regarding ability to perform an activity, hydration habits, mood, or other factors, which the device, processor, or controller can use to adjust activity motions.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. Additionally, the term “comprising” is used throughout to mean including at least the recited feature(s) such that any greater number of the same feature or additional types of other features are not precluded. To the extent any of the materials incorporated herein by reference conflict with the present disclosure, the present disclosure controls. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for monitoring and providing feedback regarding motion of a mobile electronic device associated with motion of a user of the device, the method comprising:
displaying, on the device, a training motion for a user to perform;
sensing, using a sensor of the device, first actual motion of the user;
storing, in a memory, data relating to topography of the user's body based on the first actual motion;
displaying, on the device, a first activity motion for a user to perform, the first activity motion being different from the training motion;
sensing, using a sensor of the device, second actual motion of the user;
storing, in the memory, data relating to the second actual motion of the user;
comparing the data relating to the second actual motion of the user to the first activity motion; and
based on the comparison, providing feedback regarding accuracy of the motion.

2. The method of claim 1, wherein using a sensor of the device comprises using an accelerometer or a gyroscope.

3. The method of claim 1, wherein providing feedback comprises causing a display of the device to show a graphical representation of accuracy of the motion.

4. The method of claim 3, wherein causing the display to show a graphical representation of performance comprises causing the display to show a score based on the comparison.

5. The method of claim 1, wherein providing feedback comprises causing a speaker of the device to output a noise or causing a vibration unit to vibrate.

6. The method of claim 1, further comprising displaying, on the device, a second activity motion for a user to perform, the second activity motion being different from the first activity motion and the training motion, wherein the second activity motion is based at least in part on the comparison of the data relating to the first actual motion of the user to the second actual motion.

7. A method for monitoring and providing feedback regarding motion of a user of a portable electronic device, the method comprising:
displaying, on the device, an activity motion for a user to perform;
sensing, via the device, the actual motion of the user;
storing, in a memory, data relating to the actual motion of the user;
comparing the data relating to the actual motion of the user to the activity motion; and
based on the comparison, providing feedback regarding accuracy of the actual motion.

8. The method of claim 7, wherein displaying the activity motion comprises displaying a target for a user's finger to reach or a movement for a user's finger to perform.

9. The method of claim 8 wherein the activity motion comprises a first activity motion, and wherein the method further comprises displaying a second activity motion that comprises a larger or smaller range of motion based on the comparison.

10. The method of claim 9, wherein displaying the second activity motion comprises displaying a smaller range of motion if the comparison corresponds to the user missing the target or failing to perform the movement.

11. A computer-readable storage medium storing instructions that, when executed, cause a mobile electronic device to perform a process for monitoring and providing feedback regarding motion of the device associated with motion of a user of the device, the process comprising:
displaying, on the device, a training motion for a user to perform;
sensing, using a sensor of the device, first actual motion of the user based on a user's motion of the device;
storing, in a memory, data relating to topography of the user's body based on the first actual motion;
displaying, on the device, an activity motion for a user to perform, the activity motion being different from the training motion;
sensing, using the sensor of the device, second actual motion of the user;
storing, in the memory, data relating to the second actual motion of the user;
comparing the data relating to the second actual motion of the user to the activity motion; and
based on the comparison, providing feedback regarding accuracy of the actual motion.

12. The computer-readable storage medium of claim 11, wherein sensing second actual motion of the user comprises using an accelerometer or a gyroscope.

13. The computer-readable medium of claim 11, wherein providing feedback comprises causing a display of the device to show a graphical representation of accuracy of the motion.

14. A method for monitoring and providing feedback regarding posture of a user of a portable electronic device, the method comprising:
periodically sampling a sensor in a device to determine the orientation of the device;
determining a proportion of time the device is in an orientation in which the device is oriented at an angle between 70 and 100 degrees relative to horizontal; and
periodically providing feedback regarding the proportion of time, wherein providing feedback comprises displaying, on a display of the device, information related to the proportion of time.

15. The method of claim 14, wherein periodically sampling a sensor comprises periodically receiving data, in a processor or memory, from an accelerometer or a gyroscope.

16. A system for monitoring and providing feedback regarding motion of a user of a device, the system comprising:
one or more processors; and
one or more memories storing instructions that, when executed by the one or more processors, cause the computing system to perform a process comprising:
displaying, via a display of the device, a training motion for a user to perform;
sensing, via one or more sensors of the device, a first actual motion of the user;
storing first data representative of the first actual motion;
displaying, via the display, an activity motion for a user to perform, wherein the activity motion is based at least in part on the first actual motion;
sensing, via the one more sensors of the device, a second actual motion of the user;
storing second data representative of the second actual motion;
comparing the first data to the second data; and
based on the comparison, providing feedback regarding accuracy of the second actual motion.

17. The system of claim 16, wherein the activity motion comprises a first activity motion, and wherein the instructions further comprise displaying, via the display, a second activity motion that comprises a larger or smaller range of motion based on the comparison.

18. The system of claim 17, wherein displaying the second activity motion comprises displaying a smaller range of motion if the comparison corresponds to the accuracy of the second actual motion being below a selected value.

19. The system of claim 16, wherein the one or more sensors comprises an accelerometer or a gyroscope.

20. The system of claim 16, wherein providing feedback comprises displaying graphical information on the display or providing an audio signal output from a speaker of the device.
